(19) United States
(12) Patent Application Publication
(10) Pub. No.: US 2007/0006878 A1
(43) Pub. Date: Jan. 11, 2007

(54) Capnographic-oxygenating oro-fiberscopic biteblock

(76) Inventors: Mark F. Mackey, Dallas, TX (US); James M. Rich, Rowlett, TX (US)

Correspondence Address: Michael L. Diaz, Suite 200, 555 Republic Drive, Plano, TX, 75074, US

(21) Appl. No.: 11/175,566

(22) Filed: Jul. 6, 2005

(51) Int. Cl. A61M 16/00

(52) U.S. Cl. 128/200.26

(57) ABSTRACT

The present invention is an oro-fiberscopic biteblock. The biteblock is utilized during oral fiberscopic procedures. The biteblock includes a main structure having an orifice sized to accommodate entry of a fiberscope, such as an endoscope, through the orifice. The biteblock includes an extension extending inward from the main structure when positioned within the mouth of a patient. On each side of the orifice is a loop for handling and positioning the biteblock within the patient's mouth. The biteblock includes an exhalation tube running from the extension to a monitoring device which allows monitoring of the patient's expelled gases. In addition, an inhalation tube may be used to provide supplemental oxygen to the patient. The biteblock is positioned in the mouth of the patient with the mouth of the patient surrounding the extension. The tubes include openings which are located on the extension and lie in the interior of the mouth to provide monitoring of uncontaminated gasses expelled by the patient.

### BACKGROUND OF THE INVENTION

1. Field of the Invention

This invention relates to medical devices. Specifically, the present invention relates to a biteblock for use with an oral fiberscope, transesophageal echocardiography probe, or esophageal ultrasound probe, such as an endoscope.

2. Description of the Related Art

The monitoring of a patient's respiratory status during various medical procedures is very important to determine the status of the patient. Intravenous sedation is often used during oral fiberscopic procedures. Because intravenous anesthesia is used in sedating the patient, monitoring of a patient's ventilation is critical to the well-being of the patient, as well as helping the caregiver to make clinical decisions regarding patient care. For thorough monitoring of a patient's ventilation, the monitoring of a patient's exhaled carbon dioxide should be measured using an electronic carbon dioxide monitor (capnograph). However, existing devices for use in measuring end-tidal carbon dioxide for monitoring ventilation during an oral fiberscopic procedure, have been limited at best. For example, most patients receiving sedation to facilitate endoscopic procedures normally have monitoring of cardio-respiratory parameters, before, during and after the administration of any sedation or anesthetic, however, currently this monitoring does not routinely include measuring the patient's exhaled carbon dioxide during the procedure. Exhaled carbon dioxide provides an early warning system to show when the patient's breathing is depressed or stops. Electronic monitoring is an adjunct to continuous clinical assessment by a well-trained and vigilant assistant. Commonly used monitoring equipment of patients undergoing endoscopic procedures includes pulse-oximetry, single lead continuous ECG monitoring, automated sphygmomanometry, and combination units incorporating all three of these devices. The increased use of intravenous propofol to facilitate endoscopy has also introduced several less familiar monitoring devices including end-tidal carbon dioxide monitoring, transcutaneous carbon dioxide monitoring, bispectral index monitoring (BIS), and modified electroencephalography.

To complicate matters, conscious sedation is routinely performed by non-anesthesia providers such as nurses for gastroenterology procedures and respiratory therapists for broncoscopic procedures. The operator of the scope, normally a physician, is not allowed to also be the person sedating and monitoring the patient. In addition, end-tidal carbon dioxide monitoring is also difficult to utilize because the clinical demands of oral procedures to do not lend themselves to easy monitoring of exhaled carbon dioxide. To monitor the ventilation of a patient, a respiratory rate monitor which is incorporated into the EKG monitor system and senses chest wall movement is utilized. However, the chest wall can be moving and air may or may not be moving though the airway (e.g., rocking boat motion of chest wall with obstructed airway).

As discussed above, oxygenation may also be monitored using pulse oximetry. Pulse oximeters use transcutaneous measurement of dual light wavelengths to calculate arterial oxygen saturation of the hemoglobin using a proprietary algorithm. Oxygen saturation is determined from these light waves. The oxygen saturation is known as Sp O2³. However, pulse oximetry suffers from the disadvantage of having a lag time, which can be very dangerous for the patient. For example, even though the patient's breathing slows down (respiratory depression) or stops (respiratory arrest), the pulse oximeter reading does not immediately show a decrease in the patient's oxygenation because the pulse oximetry reading stays elevated or normal until the hemoglobin begins to desaturate. If the patient is breathing supplemental oxygen, the patient may not be breathing for as long as several minutes before the oxygen saturation drops. Thus, the patient can stop breathing or decrease their breathing (hypoventilation) and the oxygen saturation can stay elevated even though the person is trending toward respiratory depression or respiratory failure. Therefore, pulse oximetry is a late indicator of respiratory depression. Not until the person has stopped breathing for several minutes will the pulse oximeter's reading begin to fall.

Automated sphygmomanometers are devices which intermittently measure and continuously display the patient's blood pressure. The oscillometric method is the most commonly employed technique for automatic blood pressure determination. In this method, the cuff is initially inflated above the systolic blood pressure. During deflation, a sensor located in the monitor detects air pressure fluctuations in the cuff. These pressure fluctuations correspond to arterial volume changes that occur because of pulsatile flow of blood. The pressure at which the oscillations peak is proportional to the mean arterial pressure. From the increasing and decreasing magnitude of these oscillations, the device uses algorithms to calculate the systolic and diastolic pressures. In addition to displaying the blood pressure, most devices calculate and display the pulse rate. The automatic blood pressure monitor plays no role in monitoring the patient's respiratory status.

ECG monitors provide a continuous display single lead ECG and can be used in conjunction with pulse oximeters and blood pressure monitors to provide real-time information regarding a patient's cardiac status (i.e. heart rate and heart rhythm) but not their respiratory status. Compact and lightweight ECG monitors are now commercially available. Available options include rate and arrhythmia alarms, filters to prevent loss of waveform during electrocautery, and printout capabilities. Various combinations of ECG, pulse oximeters, and automatic sphygmomanometers are available as compact single units.

Capnography uses infrared spectroscopy to continuously track the absorption peak of carbon dioxide at 4200 nanometers. This provides a real time graphic assessment of respiratory activity. In a non-closed system (i.e., non-endotracheal intubation), the real time graphic assessment of respiratory activity is used to augment visual assessment of the patient's ventilatory status. Both transcutaneous and end-tidal carbon dioxide monitoring are available. End-tidal carbon dioxide monitoring is accomplished by the continuous sampling of carbon dioxide from within an endotracheal tube, a face mask or at the level of a specially modified nasal cannula prong. One of the most common types of devices utilized in the non-intubated patient for monitoring ventilation is the nasal cannula. The nasal cannula has two prongs affixed to the nasal openings of the patient and connected to an oxygen tubing that provides supplemental oxygen to the patient. Incorporated into this tubing is another sampling tube which monitors the exhaled carbon dioxide as the patient breathes. However, this carbon dioxide monitoring is more qualitative rather than quantitative because the carbon dioxide is measured outside the patient's body and mixes with and is diluted by room air which decreases the accuracy of the capnographic waveform and the numeric reading. Thus, a digital readout from the nasal cannula is far more likely to create false positives (which show the patient is not ventilating when, in fact, the patient is ventilating). In addition, during oral fiberscopic procedures, the nasal cannula is awkward to use because it can be easily dislodged by the operator of the endoscope or oral probe. Since tubes originate from the nasal cannula and run directly above the mouth or cover the upper lip of the patient, it is often difficult to use an oral fiberscope without dislodging the nasal cannula. A nasal cannula having a carbon dioxide monitor is also known as a salter device.

Oximetry and capnography both play key roles in the monitor of patients receiving intravenous sedation or general anesthesia. Modern capnographs also provide immediate recognition that apnea has occurred or that the respirations are depressed. Carbon dioxide monitoring is also useful in sedated patients but usually requires some form of modification of a standard oxygen delivery system. A most commonly used method is to attach the carbon dioxide sampling line in the hub of a mixing cannula which is inserted into a perforation in a facemask. However, the use of a facemask in endoscopic procedures is just not practical because the facemask obstructs the mouth and interferes with the operator of the endoscope.

Bispectral index monitoring of sedation (BIS) has been used during gastrointestinal endoscopy. BIS employs a complex mathematical evaluation of relevant, descriptive electroencephalographic parameters of the frontal cortex corresponding to various levels of sedation. Using a specialized analysis of electroencephalogram (EEG) signals, BIS translates sedation depth into a numeric scale. However, BIS essentially provides no measurement of a patient's carbon dioxide level during ventilation or respiratory status.

It would be advantageous to have a device for use in endoscopic or other oral fiberscopic procedures which provides an accurate monitoring of a patient's carbon dioxide level while simultaneously providing supplemental oxygen to a patient.

Thus, it would be a distinct advantage to have a simple and effective monitoring device which does not obstruct the mouth of a patient, and therefore not interfere with the operator of the endoscope. In addition, it would be a distinct advantage to have a device which provides accurate carbon dioxide measurements or other gas exhalation measurements from a patient. It is an object of the present invention to provide such an apparatus.

### SUMMARY OF THE INVENTION

In one aspect, the present invention is a biteblock for use in an oral fiberscopic procedure upon a patient. The biteblock includes a main structure having an extension surrounding an orifice. The orifice is sized to accommodate entry of a fiberscope through the orifice. The biteblock also includes an exhalation tube attached to the main structure. The tube has an opening affixed on the main structure. The biteblock is positioned within the mouth of the patient to keep the mouth open during the fiberscopic procedure. The exhalation tube opening lies within the mouth of the patient. Gases expelled by the patient are then collected through the exhalation tube.

In another aspect, the present invention is a biteblock for use in an oral fiberscopic procedure upon a patient. The biteblock includes a main structure having an extension surrounding an orifice. The orifice is sized to accommodate entry of a fiberscope through the orifice. The biteblock includes an exhalation tube attached to the main structure. The tube has an opening leading from the main structure to a monitoring device. The monitoring device is used to measure gas expelled from the patient. The biteblock is used by positioning it within the mouth of the patient to keep the mouth open. The exhalation tube opening lies within the mouth of the patient and gases expelled by the patient are collected through the exhalation tube and measured by the monitoring device.

In still another aspect, the present invention is a biteblock for use in an oral fiberscopic procedure upon a patient. The biteblock includes a main structure having an extension surrounding an orifice. The orifice is sized to accommodate entry of a fiberscope through the orifice. An exhalation tube is attached to the main structure. The exhalation tube has an exhalation opening affixed to the main structure. In addition, an inhalation tube is attached to the main structure. The inhalation tube has an inhalation opening affixed upon the main structure. A monitoring device is connected to the exhalation tube and used to measure gases collected through the exhalation tube. The biteblock is positioned within the mouth of the patient to keep the mouth open during the fiberscopic procedure. The exhalation tube opening lies within the mouth of the patient and gases expelled by the patient are collected through the exhalation tube and measured by the monitoring device. In addition, the inhalation tube provides supplemental oxygen to the patient.

### BRIEF DESCRIPTION OF THE DRAWINGS

FIG. 1 is a front perspective view of a biteblock positioned in a mouth of a patient in the preferred embodiment of the present invention;

FIG. 2 is a front perspective view of the biteblock of FIG. 1 removed from the patient's mouth in the preferred embodiment of the present invention;

FIG. 3 is a rear view of the biteblock removed from the patient's mouth of FIG. 1;

FIG. 4 is a top view of the biteblock of FIG. 1 removed from the patient's mouth;

FIG. 5 is a front perspective view of a biteblock removed from a patient in an alternate embodiment of the present invention;

FIG. 6 is a top view of the biteblock of FIG. 5 in the alternate embodiment of the present invention; and

FIG. 7 is a rear perspective view of the biteblock of FIG. 5 in the alternate embodiment of the present invention.

### DESCRIPTION OF THE INVENTION

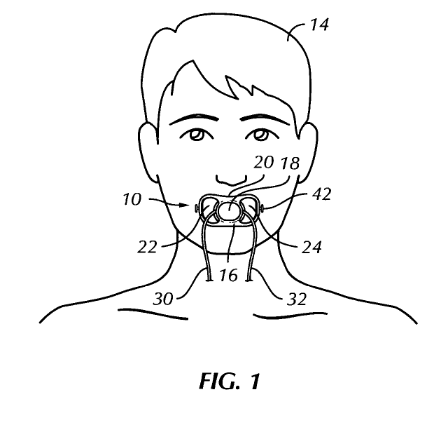

A capnographic-oxygenating oro-fiberscopic biteblock is disclosed. FIG. 1 is a front perspective view of a biteblock 10 positioned in a mouth of a patient 14 in the preferred embodiment of the present invention. The biteblock includes a main structure 16 having an opening 18 and an extension 20 extending from the main structure. Upon either side of the main structure are loops 22 and 24. The biteblock includes two tubes leading to the main structure. In addition, there is an inhalation tube 30 and an exhalation tube 32.

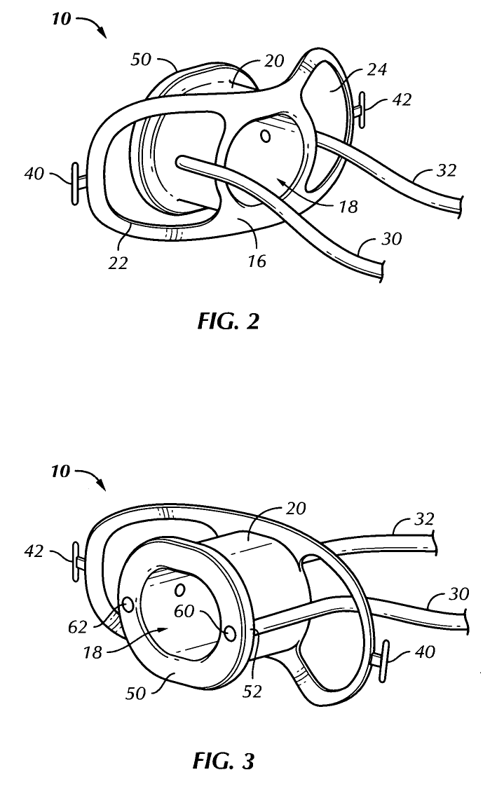

FIG. 2 is a front perspective view of the biteblock 10 of FIG. 1 removed from the patient's mouth in the preferred embodiment of the present invention. Each loop may optionally include strap holders 40 and 42. These strap holders allow a rubber strap (not shown) with holes to be used to cinch the biteblock around the patient's head to keep the bite block in place. The loops are sized and configured to allow manipulation of the biteblock by an monitoring technician upon the patient. The inhalation tube 30 and the exhalation tube 32 are preferably fused into a perimeter border 50 surrounding the opening 18 of the main structure. In the preferred embodiment of the present invention, the tubes are positioned approximately midway down upon the perimeter border. However, in alternate embodiments of the present invention, the tubes may be positioned anywhere on the biteblock which allows gaseous substances to be fed into the patient's mouth (e.g., oxygen) or out from the patient's mouth (e.g., carbon dioxide). The tubes may be integrated into the perimeter border or attached to the side of the perimeter border.

FIG. 3 is a rear view of the biteblock 10 removed from the patient's mouth of FIG. 1. FIG. 4 is a top view of the biteblock 10 of FIG. 1 removed from the patient's mouth. The main structure includes the extension 20 providing an extension of the perimeter border. The extension includes a curved wall section 52 surrounding the opening 18 and forming an integral part of the extension 20. The curved wall preferably allows the patient's teeth/outer portion of the mouth to rest against the perimeter border and extension, thereby providing an unobstructed opening to the patient's mouth.

The inhalation tube 30 may provide a gaseous substance to the patient 14. For example, oxygen may be provided to the patient. However, any gaseous substance may be provided to the patient. The exhalation tube 32 gathers gases, such as carbon dioxide, when the patient exhales. As the patient exhales, the exhaled gases exit through the exhalation tube leading to a gas monitor (e.g., CO2 monitor, which is not shown). The inhalation tube includes an orifice 60 and the exhalation tube includes an orifice 62. The orifices are located on the perimeter border. In the preferred embodiment of the present invention, the orifices are elliptical in shape. The elliptically shape provides a relatively large surface area for sampling expelled gases, such as carbon dioxide and decreases the likelihood of the orifices being blocked by oral secretions form the patient. The elliptical shape of the sampling port may increase the size of the port, thereby making it less likely to be clogged or occluded by secretions from the patient or by touching it with the fiberscope. The inclusion of 2 or 3 sampling ports may further ensure that occlusion does not occur easily.

With reference to FIGS. 1-4, the operation of the biteblock 10 will now be explained. When desired during an endoscopic procedure, the biteblock is positioned within the mouth 12 of the patient 14. Preferably, the teeth or mouth of the patient rest against the wall section 52. This protects the expensive fiberscope by preventing the patient from biting down upon the fiberscope. The biteblock is positioned with the orifices within the mouth of the patient to allow an undiluted concentrated collection of the exhalation gases (carbon dioxide) from the patient without any contamination (e.g., dilution of gases from occurring) outside of the mouth. The biteblock keeps the opening of the mouth in an open position. As required, an endoscope or other oral scoping device is inserted through the opening 18 into the mouth of the patient.

With the biteblock in place, the patient is optionally provided with oxygen through the inhalation tube 30. In addition, the gases exhaled by the patient 14 are collected by the exhalation tube 32 and measured by a gas monitoring device (e.g., infra-red aspirating carbon dioxide monitor). The carbon dioxide monitor may continuously draw a sample from exhaled gas and measure PeCO₂, which can then be drawn as a time-related waveform. In particular, the carbon dioxide exhaled by the patient is measured by the monitoring technician or other individual to allow proper monitoring of the patient.

The biteblock 10 enables a very accurate monitoring of exhaled gases from the patent without having contamination or dilution from other sources. Specifically, the accuracy is obtained because exhaled gases (e.g., end tidal gases—those collecting at the end point of exhalation) from the patient are obtained within the oral cavity of the mouth rather than outside of the mouth. For example, when carbon dioxide is measured from inside the mouth before it is diluted by ambient room care, a better capnographic wave form can be generated. The waveform produced by salter monitors (i.e., existing devices) are far more sloped whereas the waveform produced by a carbon dioxide monitor connected to a closed endotracheal tube as shown in the present invention provide a classic and far more accurate “rectangular formation” waveform. Measuring the gas from the mouth will cause the waveform to be more like the waveform from an endotracheal (more accurate) than the waveform from a Salter monitor (less accurate). In addition, oxygen or other gases may be provided to the patient without causing drying mucosa within the nasal cavity. In existing systems, a nasal cannula is inserted in the nasal passages with oxygen being provided through the nose. This process tends to dry the nasal mucosa by oxygen being administered within the nose. However, the present invention provides oxygen though the mouth, which is very moist from secretion of saliva and where mucosal drying is not a factor. In addition, in the preferred embodiment of the present invention, the biteblock includes orifices 60 and 62 which are elliptical in shape, thereby providing a greater surface area for intake and collection of gasses as well as decreasing the possibility of blockage of the orifices by oral secretions.

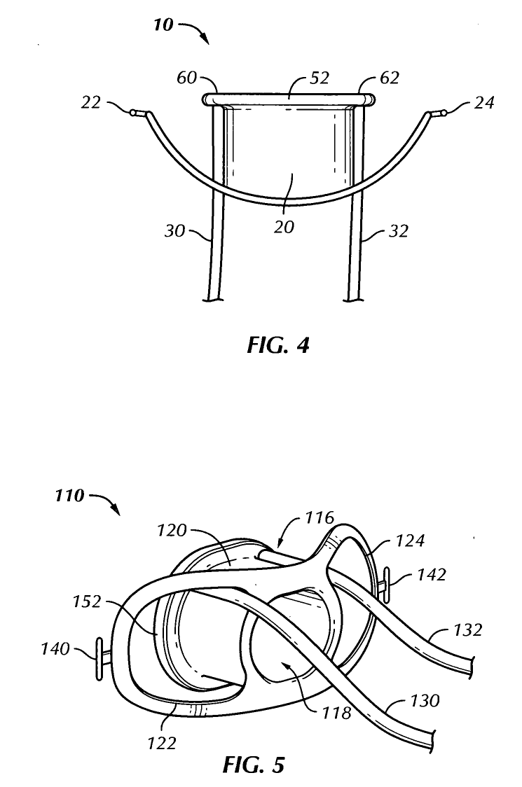

FIG. 5 is a front perspective view of a biteblock 110 removed from a patient in an alternate embodiment of the present invention. The biteblock 110 is very similar to the biteblock 10 having a main structure 116, an opening 118, an extension 120 having loops 122 and 124, and strap holders 140 and 142. In addition, the biteblock 110 also includes a perimeter border 150 and a wall section 152. In addition, the biteblock also includes an inhalation tube 130 and an exhalation tube 132. On the backside of the biteblock, the inhalation tube 130 is attached to the perimeter border 150 at the orifice 160. Likewise, on an opposite side of the perimeter border is the orifice 162 leading to the exhalation tube.

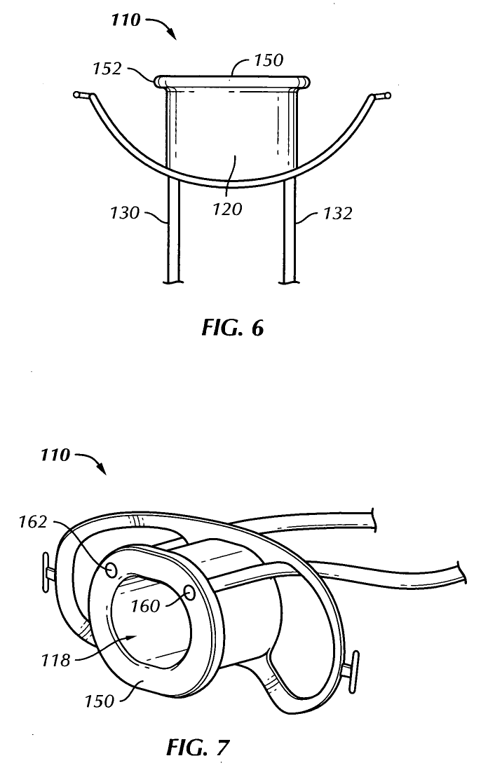

FIG. 6 is a top view of the biteblock 110 of FIG. 5 in the alternate embodiment of the present invention. FIG. 7 is a rear perspective view of the biteblock 110 of FIG. 5 in the alternate embodiment of the present invention. The biteblock 110 differs from the biteblock 10 in that the tubes are located on a top portion of the perimeter border 150.

The present invention may be used for any procedure requiring the insertion of a device into the mouth of a patient. For example, the present invention may be used for pulmonary procedures which utilize a bronchoscope, which is inserted through the bite block and into the upper airway to look at the airway and lungs. The present invention may also be used for oral endoscopy which utilizes an endoscope. The endoscope is inserted through the bite block within the opening of the biteblock and into the esophagus to look at the esophagus and upper gastrointestinal track.

Additionally, the present invention may be used for transesophageal echocardiography (TEE) (used by cardiologists, anesthesiologists and other practitioners) which involves the placement of a probe into the esophagus to measure heart function using ultrasonic energy. TEE probes are similarly used through the mouth in a similar fashion as endoscopic and broncoscopic probes of the patient. The biteblock may also be used for endoscopic ultrasound which is performed by gastroenergologists to evaluate gastric organs within the patient.

In addition, the present invention may also be used in a sedated patient to keep the upper airway open (in a similar fashion as an oral airway does) and simultaneously monitor the exhaled carbon dioxide of the patient and simultaneously administer oxygen to a patient who was having their trachea intubated with an endotracheal tube via the use of a flexible fiber optic laryngoscope. The present invention may be employed as an oral airway that is used to keep the mouth open (in a similar manner as the biteblock), but also helps keep the upper airway open. Thus, the present invention may be utilized as a biteblock, oral airway, an oxygenator, and a carbon dioxide monitor. When the present invention is used as an oral airway, the biteblock may be elongated and curved in the shape of the upper airway and allowed to extend back into the upper airway so that the distal tip actually lies in the oropharynx and presses the tongue forward. The oral airway/biteblock would simply have the same monitoring tubes and orifices and oxygen tubes and orifices built into walls of the device. Thus, the present invention may be used in fiber optic assisted tracheal intubation for use as an oral airway (e.g., Ovasappian Airway or Burman Airway).

It should be understood that the tubes may be positioned anywhere on the biteblock which enables the collection and distribution of gas to and from the patient. Additionally, although two tubes are depicted, the biteblock may be used in conjunction with one or more tubes. The tubes may be formed into the perimeter of the biteblock or detachably attached to the biteblock. Additionally, the tubes may be run separately or together prior to attaching to the biteblock.

The present invention provides a revolutionary apparatus for monitoring anesthesia care and conscious sedation patients. The present invention may be used by either anesthesia or non-anesthesia providers who need assistance in providing quality monitoring of the patient during procedures involving intravenous sedation.

While the present invention is described herein with reference to illustrative embodiments for particular applications, it should be understood that the invention is not limited thereto. Those having ordinary skill in the art and access to the teachings provided herein will recognize additional modifications, applications, and embodiments within the scope thereof and additional fields in which the present invention would be of significant utility.

Thus, the present invention has been described herein with reference to a particular embodiment for a particular application. Those having ordinary skill in the art and access to the present teachings will recognize additional modifications, applications and embodiments within the scope thereof.

It is therefore intended by the appended claims to cover any and all such applications, modifications and embodiments within the scope of the present invention.

1. A biteblock for use in an oral fiberscopic procedure upon a patient, the biteblock comprising:
a main structure having an extension surrounding an orifice, the orifice sized to accommodate entry of a fiberscope through the orifice; and
at least one exhalation tube attached to the main structure, the tube having an opening affixed upon the main structure;
whereby the biteblock is positioned within the mouth of the patient to keep the mouth open during the fiberscopie procedure, the exhalation tube opening lying within the mouth of the patient and gases expelled by the patient are collected through the exhalation tube.

2. The biteblock of claim 1 further comprising an inhalation tube attached to the main structure, the inhalation tube having an opening affixed upon the main structure:
whereby the inhalation tube having an opening lies within the mouth of the patient and provides a gaseous substance to the patient through the inhalation tube.

3. The biteblock, of claim 2 wherein the gaseous substance is oxygen.

4. The biteblock of claim 1 further comprising a premier border position on an inner surface of the extension, the premier border being positioned within the mouth during use of the biteblock and wherein the exhalation tube is attached to the perimeter border.

5. The biteblock of claim 4 wherein the exhalation tube is affixed to a top portion of the perimeter border.

6. The biteblock of claim 4 further comprising an inhalation tube attached to the main structure, the inhalation tube having a second opening affixed upon the perimeter border,
whereby the inhalation tube opening lies within the mouth of the patient and provides a gaseous substance to the patient through the inhalation tube.

7. The biteblock of claim 6 wherein the inhalation tube is affixed to a top portion of the perimeter border.

8. The biteblock of claim 1 wherein the opening of the exhalation is elliptical in shape.

9. The biteblock of claim 1 wherein the exhalation tube is connected to a gas monitor providing an analysis of the expelled gases collected through the exhalation tube.

10. The biteblock of claim 9 wherein the gas monitor is a carbon dioxide gas monitor.

11. The biteblock of claim 1 wherein the exhalation tube is detachable from the biteblock.

12. The biteblock of claim 1 wherein the biteblock allows entry through the opening by an endoscope.

13. The biteblock of claim 1 wherein the biteblock allows entry through the opening by a bronchoscope.

14. The biteblock of claim 1 wherein the biteblock allows entry through the opening by a transesophageal probe.

15. The biteblock of claim 1 further comprising a first loop attached to a first side of the orifice and a second loop attached to a second opposite side of the orifice, the first and second loops providing handling of he biteblock and holding the biteblock in place within the mouth of the patient.

16. The biteblock of claim 1 wherein the biteblock is sized and shaped to act as an oral airways.

17. A biteblock for use in an oral fiberscopic procedure upon a patient, the biteblock comprising:
a main structure having an extension surrounding an orifice, the orifice sized to accommodate entry of a fiberscope through the orifice;
at least one exhalation attached to the main structure, the tube having an opening affixed upon the main structure; and
whereby the biteblock is positioned within the mouth of the patient to keep the mouth open, the explanation exhalation tube opening lying within the mouth of the patient and gases expelled by the patient are collected through the exhalation tube and measured by the monitoring device.

18. The biteblock of claim 17 further comprising an inhalation tube attached to the main structure, the inhalation tube having an inhalation opening affixed upon the main structure:
whereby the inhalation tube opening lies within the mouth of the patient and provides a gaseous substance to the patient through the inhalation tube.

19. The biteblock of claim 17 wherein the monitoring device measures carbon dioxide expelled from the patient.

20. A biteblock for use in an oral fiberscopie procedure upon a patient, the biteblock comprising:
a main structure having an extension surrounding an orifice, the orifice sized to accommodate entry of a fiberscope through the orifice;
an exhalation tube attached to the main structure, the exhalation tube having an exhalation opening affixed upon the main structure;
a inhalation tube attached to the main structure, the inhalation tube having an inhalation opening affixed upon the main structure, and
a monitoring device connected to the exhalation tube, the monitoring device measuring gases collected through the exhalation tube;
whereby the biteblock is positioned within the mouth of the patient to keep the mouth open during the fiberscopic procedure, the exhalation tube opening lying within the mouth of the patient and gases expelled by the patient are collected through the exhalation tube and measured by the monitoring device and the inhalation tube providing supplemental oxygen to the patient.
